(19) United States
(12) Patent Application Publication
(10) Pub. No.: US 2009/0111741 A1
(43) Pub. Date: Apr. 30, 2009

(54) METHOD FOR TREATING AND/OR PREVENTING DRUG SEEKING BEHAVIOR

(76) Inventors: Jane V. Aldrich, Lawrence, KS (US); Kshitij A. Patkar, Lawrence, KS (US); Jay McLaughlin, Natick, MA (US)

Correspondence Address: Workman Nydegger;1000 Eagle Gate Tower, 60 East South Temple, Salt Lake City, UT, 84111, US

(21) Appl. No.: 12/248,252

(22) Filed: Oct. 9, 2008

Related U.S. Application Data
(60) Provisional application No. 60/978,906, filed on Oct. 10, 2007.
(60) Provisional application No. 61/021,985, filed on Jan. 18, 2008.

(51) Int. Cl. A61K 38/00; A61K 38/12; C12N 5/00; A61P 25/00

(52) U.S. Cl. 514/9; 514/12; 435/375

(57) ABSTRACT

A dynorphin-A analog can be used for treatment, inhibition, and/or prevention of cocaine seeking behavior, and or the drug seeking behavior for a cocaine derivative or other structurally related substance. The dynorphin-A analog can be a cyclic dynorphin-A analog having sufficient systemic stability that crosses the blood-brain barrier so as to be active in the brain at kappa-opioid receptors (KOR) as an antagonist. Such activity at a KOR as an antagonist can be useful for cocaine management and reducing the desire, such as stress-related desires, for use of cocaine, crack, or the like. The KOR antagonist can be [N-benzylTyr¹,cyclo(D-Asp⁵,Dap⁸)]Dyn A-(1-11) amide, salt thereof, prodrug thereof, and/or derivative thereof.

### CROSS-REFERENCE TO RELATED APPLICATIONS

The present invention claims the benefit of U.S. Provisional Application Ser. No. 60/978,906, filed Oct. 10, 2007, and U.S. Provisional Application Ser. No. 61/021,985, filed Jan. 18, 2008, which applications are incorporated herein by specific reference in their entirety.

The U.S. Government has a paid-up license in this invention and the right in limited circumstances to require the patent owner to license others on reasonable terms as provided for by the terms of grant R01 DA018832 awarded by the National Institute of Health's National Institute on Drug Abuse.

### BACKGROUND

Cocaine use and addiction is a world wide problem that has serious social, mental, and physical consequences. While various forms of prevention and/or treatment of cocaine addiction have been attempted, there currently is no drug approved to treat this addiction. For example, small molecules have been used as drugs to decrease the physical and/or mental conditions associated with cocaine addiction. However, many small molecules with bioactivity have negative side effects due to the ability of the small molecule to not only interact with the proper receptor(s) associated with cocaine addition (e.g., target receptors), but to also cross-interact with unintended receptors (e.g., non-target receptors). It is the activity with non-target receptors that typically causes such negative side effects.

Also, small molecule antagonists of receptors can have exceptionally long activity. While being selective, such long activity can also cause side effects and have complications that preclude such small molecules from being therapeutically useful.

Therefore, it would be advantageous to have a bioactive agent for use in treating and/or preventing cocaine addiction that does not have substantial negative side effects. More particularly, it would be beneficial to have a polypeptide bioactive agent that has preferentially targets receptors associated with cocaine addiction over non-target receptors and has a suitable length of activity.

### SUMMARY

In one embodiment, the present invention includes a method for antagonizing kappa-opioid receptors present in human or animal tissue in vitro or in vivo. Such a method can include administering an effective amount of a polypeptide kappa-opioid receptor (KOR) antagonist to a subject such that a sufficient amount of the polypeptide KOR antagonist is active in the brain for antagonizing kappa-opioid receptors.

In one embodiment, the polypeptide KOR antagonist can be administered systemically. This can include subcutaneous, intravenous, inhalation, and the like.

In one embodiment, the polypeptide KOR antagonist can be a dynorphin-A analogue. This can include the KOR antagonist having a cyclic peptide portion. Also, the KOR antagonist can be [N-benzylTyr¹,cyclo(D-Asp⁵,Dap⁸)]Dyn A-(1-11) amide, salt, prodrug, or derivative thereof. Dap is 1,2,-diaminoproprionic acid.

In one embodiment, the polypeptide KOR antagonist is selective for KOR over other opioid receptors. This includes the KOR antagonist being selective for KOR over mu-opioid and delta-opioid receptors.

In one embodiment, the KOR antagonist can be more effective compared to small molecule KOR antagonists. It can also include the KOR antagonist being more effective as a KOR antagonist compared to JDTic. Also, the KOR antagonist can be a cyclic peptide that is more stable than a linear peptide. Moreover, the KOR antagonist can be capable of crossing the blood brain barrier.

In one embodiment, the effective amount of KOR antagonist is sufficient for treating, inhibiting, and/or preventing a condition. Such a condition can be selected from at least one of the following: depression; drug seeking behavior; opiate seeking behavior or addiction; methamphetamine seeking behavior or addiction; alcohol seeking behavior or addiction; nicotine seeking behavior or addiction; ecstasy seeking behavior or addiction; or cocaine and/or cocaine derivative seeking behavior or addiction.

In one embodiment, the effective amount of KOR antagonist is sufficient for treating, inhibiting, and/or preventing stress-induced seeking behavior of an addictive substance selected from the group consisting of opiates, methamphetamines, alcohol, nicotine, ecstasy, cocaine, cocaine derivative, or combinations thereof.

In one embodiment, the present invention includes a method for treating, inhibiting, and/or preventing cocaine and/or cocaine derivative seeking behavior and/or addiction. Such a method can include identifying a person that has cocaine and/or cocaine derivative seeking behavior; and administering an effective amount of a polypeptide kappa-opioid receptor (KOR) antagonist to the person such that a sufficient amount of the polypeptide KOR antagonist is active in the brain for antagonizing KOR. The cocaine and/or cocaine derivative seeking behavior can be stress-induced.

In one embodiment, the polypeptide KOR antagonist can be administered systemically and crosses the blood brain barrier in a sufficient amount. Such systemic administration is described herein. Also, the amount of KOR antagonist administered can be insufficient for substantial interaction with other opioid receptors.

In one embodiment, the method can further include the following: providing a pharmaceutically-acceptable composition suitable for human administration that contains [N-benzylTyr¹,cyclo(D-Asp⁵,Dap⁸)]Dyn A-(1-11) amide and a pharmaceutically-acceptable carrier; and systemically administering an effective amount of [N-benzylTyr¹,cyclo(D-Asp⁵,Dap⁸)]Dyn A-(1-11) amide to the person such that a sufficient amount of [N-benzylTyr¹,cyclo(D-Asp⁵,Dap⁸)]Dyn A-(1-11) amide crosses the blood brain barrier and is active in the brain for antagonizing kappa-opioid receptors so as to inhibit stress-induced cocaine and/or cocaine derivative seeking behavior.

In one embodiment, the present invention includes a pharmaceutical composition having a kappa-opioid receptor (KOR) antagonist. Such a pharmaceutical composition can include a pharmaceutically-acceptable carrier. The pharmaceutically acceptable carrier can be suitable for any type of systemic administration, such as those described herein. Also, the pharmaceutical composition can include a therapeutically effective amount of a kappa-opioid receptor (KOR) antagonist that antagonizes a sufficient amount of KOR receptors for treating, inhibiting, and/or preventing cocaine and/or cocaine derivative seeking behavior and/or addiction.

In one embodiment, the KOR antagonist is [N-benzylTyr¹,cyclo(D-Asp⁵,Dap⁸)]Dyn A-(1-11) amide, salt thereof, prodrug thereof, and/or derivative thereof.

### FIGURES

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates the chemical structures of Dynorphin A-(1-11) amide, arodyn, and [N-benzylTyr¹,cyclo(D-Asp⁵,Dap⁸)]Dyn A-(1-11) amide.

FIG. 2 is a graph illustrating data related to [N-benzylTyr¹,cyclo(D-Asp⁵,Dap⁸)]Dyn A-(1-11) amide exhibiting KOR antagonist activity in vivo.

FIG. 3 is a graph illustrating data related to the duration of in vivo kappa-opioid receptor antagonist effects of [N-benzylTyr¹,cyclo(D-Asp⁵,Dap⁸)]Dyn A-(1-11) amide in C57B1/6J mice using the 55° C. warm-water tail-withdrawal test.

FIG. 4 is a graph illustrating data related to [N-benzylTyr¹,cyclo(D-Asp⁵,Dap⁸)]Dyn A-(1-11) amide being active after systemic delivery.

FIG. 5 is a graph illustrating data related to [N-benzylTyr¹,cyclo(D-Asp⁵,Dap⁸)]Dyn A-(1-11) amide administered subcutaneously blocking the agonist activity of intracerebroventricularly administered U50,488, and crossing the blood-brain barrier.

FIG. 6 is a graph illustrating data related to [N-benzylTyr¹,cyclo(D-Asp⁵,Dap⁸)]Dyn A-(1-11) amide being useful in inhibiting cocaine seeking behavior.

FIG. 7 is a graph illustrating data related to [N-benzylTyr¹,cyclo(D-Asp⁵,Dap⁸)]Dyn A-(1-11) amide being selective for KOR over other opioid receptors.

### DETAILED DESCRIPTION

Cocaine and its free base derivative commonly referred to as crack are highly additive drugs obtained as products extracted from the leaves of Erythroxylon coca Lam (coca leaves). The systematic name of cocaine is [1R-(exo,exo)]-3-(benzoyloxy)-8-methyl-8-azabicyclo[3.2.1]octane-2-carboxylic acid methyl ester. Cocaine is the methyl ester of benzoylecgonine and is also known as 3β-hydroxy-1αH,5α-H-tropane-2β-carboxylic acid methyl ester benzoate. Although four pairs of enantiomers are theoretically possible, only one (commonly termed l-cocaine) occurs naturally. Cocaine is structurally related to atropine (hyoscamine) and hyoscine (scopolamine) as well as some anesthetics, substances with quite different pharmacological properties.

Accordingly, the present invention can include the use of a dynorphin-A analog for treatment, inhibition, and/or prevention of cocaine seeking behavior, and or the drug seeking activity for a cocaine derivative or other structurally related substance. More particularly, the present invention relates to the use of an effective amount of a cyclic dynorphin-A analog having sufficient systemic stability that crosses the blood-brain barrier so as to be active in the brain at kappa-opioid receptors (KOR) as an antagonist. Such activity at a KOR as an antagonist can be useful for cocaine management and reducing the desire, such as stress-related desires, for use of cocaine, crack, or the like.

### I. INTRODUCTION

The ability of a kappa-opioid receptor (i.e., KOR) agonist to interact with the KOR has been contemplated as a biological process that may be used for pharmacological management of cocaine addiction. However, it has recently been determined that a small molecule KOR antagonist can also be used in the pharmacological management of cocaine addiction. Particularly, the ability of a small molecule (e.g., (3R)-7-hydroxy-N-{(1S)-1-{[(3R,4R)-4-(3-hydroxyphenyl)-3,4-dimethyl-1-piperidinyl]methyl}-2-methylpropyl}-1,2,3,4-tetrahydro-3-isoquinolinecarbox-amide, JDTic) to block the kappa-opioid receptor (e.g., KOR) as an antagonist (e.g., JDTic) has been shown to block stress-induced reinstatement of cocaine-seeking behavior has been published (Beardsley et al, Psychopharmacology, 2005, 183, 118-126).

Numerous studies have demonstrated that KOR agonists (i.e., compounds that activate the receptors) can acutely block cocaine seeking behavior, but paradoxically chronic (i.e., long term) administration of KOR agonists has been reported to potentiate drug-seeking behavior. Thus, KOR antagonists (i.e., compounds that block the activity of agonists at the receptors) may be better suited for chronic treatment of cocaine abuse.

KOR antagonists are known to be useful in treatment of opiate addition and depression; however, many polypeptide KOR antagonists suffer from being unstable, unable to cross the blood brain barrier, and unusable for systemic administration. Since small molecule KOR antagonists have been identified as potential therapeutic agents for pharmacological management of cocaine addiction, and polypeptide KOR antagonists have been identified as being unstable and unusable for systemic administration, the use of a polypeptide KOR antagonist has not previously been considered.

Recently, it has been shown that a dynorphin-A analog, arodyn, can interact with the KOR. As such, it was demonstrated that arodyn, which is not metabolically stable, can interact with the KOR and thereby can block stress-induced cocaine seeking behavior following administration directly into the brain (Eur. J. Pharmacol., 2007, 569, 84-89).

There are a few other dynorphin analogs that exhibit systemic activity, but these are KOR agonists and are not very selective for KOR over other opoid receptors such as μ-opioid receptors (MOR). The best studied dynorphin analog is a dynorphin A-(1-8) analog E-2078 ([NMeTyr¹,NMeArg⁷,D-Leu⁸]-dynorphin A-(1-8) N-ethylamide) which has been administered to humans in Japan. Another dynorphin A-(1-8) analog SK-9709 ([D-Ala²,Arg⁶(CH₂NH)Arg⁷]-dynorphin A-(1-8) amide has also been studied in vivo following different routes of administration (J. Pharmacol. Exper. Ther., 2001, 132, 1948-1956). There is also one report of longer dynorphin A-(1-13) analogs administered systemically (AAPS J., 2004, 6, e36). However, none of these dynorphin analogs have the characteristics to be useful for the chronic treatment, inhibition, and/or prevention of cocaine seeking behavior.

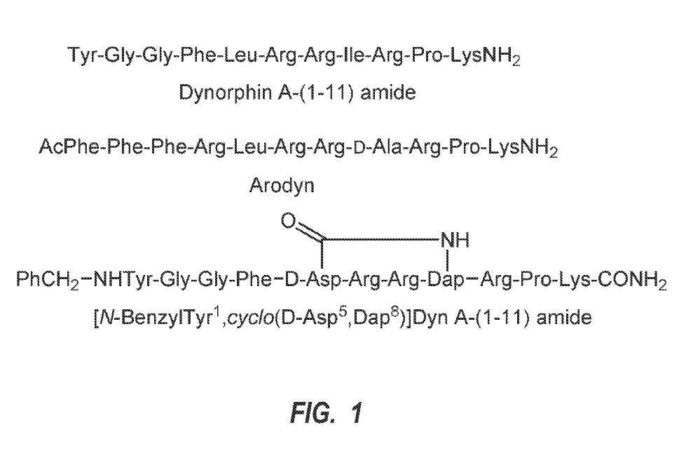

FIG. 1 shows Dynorphin A-(1-11) amide and two Dyn A-(1-11) amide analogs that were studied for their metabolic stability and ability to cross the BBB. Accordingly, two selective KOR peptide antagonists, arodyn (Ac[Phe^{1,2,3},Arg⁴,D-Ala⁸]Dyn A-(1-11) amide) and [N-benzylTyr¹,cyclo(D-Asp⁵,Dap⁸)]Dyn A-(1-11) amide, were examined for KOR antagonist activity in vivo. Both of these peptides exhibit high selectivity for KOR (Kᵢ ratios (KOR/MOR/DOR (delta opioid receptor))=1/174/583 and 1/194/>330, respectively) and nanomolar antagonist potency against KOR in vitro. However, it was found that [N-benzylTyr¹,cyclo(D-Asp⁵,Dap⁸)]Dyn A-(1-11) amide was significantly more stable and capable of traversing the blood-brain barrier (BBB).

In vitro results for [N-benzylTyr¹,cyclo(D-Asp⁵,Dap⁸)]Dyn A-(1-11) amide were as follows: binding affinity, Kᵢ, with KOR=30±2 nM, and Kᵢ ratio (KOR/MOR/DOR)=1/194/>330; antagonist activity (adenylyl cyclase assay) was K_{B}=84 nM; preliminary metabolism studies showed it was stable for at least 3 h in whole blood, and had a t_{1/2}˜70 min in rat brain homogenate

### A. DEFINITIONS

As used herein, the terms “an effective amount”, “therapeutic effective amount”, or “therapeutically effective amount” shall mean an amount or concentration of a compound according to the present invention which is effective within the context of its administration or use. Thus, the term “effective amount” is used throughout the specification to describe concentrations or amounts of compounds according to the present invention which may be used to produce a favorable change in the disease or condition treated, inhibited, or prevented, whether that change is a remission, a decrease in desire for a drug such as cocaine or in addiction characteristics, a favorable physiological result, or the like, depending upon the disease or condition treated.

As used herein, the term “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

As used herein, the term “pharmaceutically acceptable acid addition salts” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as trifluoroacetic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Groups which form pharmaceutically acceptable acid addition salts include amines, hydrazines, amidines, guanidines, substituted aryl/heteroaryl and substituted alkyl groups that carry at least a nitrogen bearing substitutent such as amino, guanidine, amidino, and the like.

As used herein, the term “coadministration” or “combination therapy” is used to describe a therapy in which at least two active compounds in effective amounts are used for the treatment, inhibition, and/or prevention of cocaine or other drug addiction, or cocaine or other drug-seeking activity, such as stress-induced drug-seeking activity. Although the term coadministration preferably includes the administration of two active compounds to the patient at the same time, it is not necessary that the compounds be administered to the patient at the same time, although effective amounts of the individual compounds will be present in the patient at the same time.

Compounds according to the present invention may be used in pharmaceutical compositions having biological/pharmacological activity for the treatment, inhibition, and/or prevention of cocaine or other drug addiction, or cocaine or other drug-seeking activity. These compositions comprise an effective amount of any one or more of the compounds disclosed herein, optionally in combination with a pharmaceutically acceptable additive, carrier, or excipient.

As used herein, the term “treating” or “treatment” of a disease, including drug addiction and drug-seeking behavior, includes: (a) preventing the disease, i.e. causing the clinical symptoms of the disease not to develop in a mammal that may be exposed to or predisposed to the disease but does not yet experience or display symptoms of the disease; (b) inhibiting the disease, i.e., arresting or reducing the development of the disease or its clinical symptoms; or (c) relieving the disease, i.e., causing regression of the disease or its clinical symptoms.

As used herein, the term “unit dosage form,” refers to physically discrete units suitable as unitary dosages for human and animal subjects, each unit containing a predetermined quantity of pharmacological agent calculated in an amount sufficient to produce the desired effect in association with a pharmaceutically acceptable diluent, carrier or vehicle.

As used herein, a “subject” or a “patient” refers to any mammal (preferably, a human), and preferably a mammal that may be susceptible to cocaine or other drug addiction, or cocaine or other drug-seeking activity. Examples of a subject or patient include a human, a non-human primate, a cow, a horse, a pig, a sheep, a goat, a dog, a cat or a rodent such as a mouse, a rat, a hamster, or a guinea pig. Generally, the invention is directed toward use with humans.

### II. ZYKLOPHIN

Accordingly, the present invention is a stable polypeptide KOR antagonist in the form of a dynorphin analog that can be used in the treatment, inhibition, and/or prevention of cocaine addiction. More particularly, the stable polypeptide dynorphin analog KOR antagonist can be [N-benzylTyr¹,cyclo(D-Asp⁵,Dap⁸)]Dyn A-(1-11) amide, which is also referred to herein as zyklophin, as shown in FIG. 1. This dynorphin analog KOR antagonist, zyklophin, can be used in the treatment, inhibition, and/or prevention of drug abuse, specifically for the treatment, inhibition, and/or prevention of stress-induced drug seeking behavior.

The dynorphin analog KOR antagonist zyklophin has been synthesized, and the in vitro characterization in cell culture has been published (J. Med. Chem. 2005, 48, 4500-4503). It has recently been demonstrated that the dynorphin analog KOR antagonist zyklophin is metabolically stable, and that it is active in the brain following systemic (e.g., subcutaneous, s.c.) administration. As such, the dynorphin analog KOR antagonist zyklophin can be administered systemically and is capable of crossing the blood brain barrier. The dynorphin analog KOR antagonist zyklophin can block (e.g., treat, inhibit, or prevent) stress-induced reinstatement of cocaine-seeking behavior following systemic administration. Thus, the polypeptide KOR antagonist zyklophin can be used for the treatment, inhibition, and/or prevention of cocaine addiction or other cocaine seeking behavior.

In one embodiment, the stable polypeptide dynorphin analog KOR antagonist can be an analog of [N-benzylTyr¹,cyclo(D-Asp⁵,Dap⁸)]Dyn A-(1-11) amide. It is common for molecular scaffolds having therapeutic potential to be used in preparing analogs thereof for screening for the same or different bioactivity. As such, the present invention is described in connection to zyklophin as a lead peptide dynorphin analog KOR antagonist; however, it is contemplated that analogs of this compound can have similar biological activity and therapeutic potential. Derivativation of molecular scaffolds is well known in the art, and such principles of derivativation can be used in preparing the analogs of [N-benzylTyr¹,cyclo(D-Asp⁵,Dap⁸)]Dyn A-(1-11) amide (e.g., zyklophin analogs). For example, any of the following derivations can be used in preparing a zyklophin analog in accordance with the present invention: substitution of a hydrogen with a halogen, alkyl (C1-C10), or the like; substitution of an alkyl with a halogen, other alkyl (C1-C10), or the like; substitution of an aryl ring carbon with a nitrogen, oxygen, or the like; substitution of an aryl ring with an alkyl (C1-C10) group; substitution of one or more amino acids with a derivative of the amino acid, other amino acid, or the like; derivatization of the “message” portion of zyklophin; derivatization of the “address” portion of zyklophin; or other derivatization. Also, zyklophin can be prepared into a pharmaceutically acceptable salt or prodrug.

It has been found that [N-benzylTyr¹,cyclo(D-Asp⁵,Dap⁸)]Dyn A-(1-11) amide (i.e., zyklophin) is very selective for KOR over other opioid receptors, especially the mu opioid receptors (i.e., μ-opioid receptors or MOR) where morphine is active. The ability of zyklophin to be highly selective for KOR over other opioid receptors provides the ability to be used as a therapeutic agent with low toxicity or other unwanted side effects. Moreover, the selectivity for a KOR can aide in FDA approval.

It has been found that zyklophin can cross the blood-brain barrier and block KOR in the brain following peripheral administration. Accordingly, zyklophin is the first peptide KOR antagonist that has been demonstrated to cross the blood-brain barrier (BBB) and block KOR in the brain following peripheral administration. The ability of zyklophin to cross the BBB allows for any type of systemic administration, such as subcutaneous administration. Accordingly, zyklophin can be administered in a manner other than, but including, intercerebroventricular administration. Moreover, the ability of zyklophin to cross the BBB allows for a myriad of formulation opportunities, where the formulation can be prepared for a suitable mode or route of administration. As a consequence of having high selectivity for KOR and BBB permeability, zyklophin is a favorable drug candidate and as a lead drug for derivatizations and analoging. Support for the benefits of zyklophin are supported in that no unwanted side affects have been observed in mice that have been administered zyklophin. The mice data is discussed in more detail herein and in the incorporated references.

It has now been found that zyklophin can have a shorter half life compared to small molecule KOR antagonists. It is expected, based on experimental data described herein and in the incorporated references, that zyklophin will not have the pharmacokinetic problems associated with small molecule KOR antagonists. For example, zyklophin can have a shorter half life compared to the exceptionally long activity of small molecule KOR antagonists in animal models (e.g., weeks to over a month after a single injection) that complicate their potential therapeutic use in humans. The shorter half life of zyklophin can be beneficial for obtaining a therapeutic composition for use in humans. In part, zyklophin is expected to have a shorter acting time period or shorter half life because of its metabolism by proteases or other degradation even though zyklophin has increased stability. By comparison, zyklophin can have a longer half life than other, less stable polypeptides, but can also have a much shorter pharmacological half life than small molecule KOR antagonists.

It has been found that zyklophin can have sufficient metabolic stability in vivo so as to be capable of systemic administration. Zyklophin has been studied for its metabolic stability and for its activity in vivo, and the results showed improved metabolic stability over other polypeptides that are KOR antagonists. To determine the metabolic stability of zyklophin, the disappearance of zyklophin was examined in both rat whole blood and brain homogenate, and by using electrospray mass spectrometry in order to quantify the amount of zyklophin remaining after certain periods of time. In contrast to other dynorphin-based kappa opioid receptor (KOR) antagonists (e.g. arodyn), zyklophin was found to be more stable in both of these physiological media. Stability data showed that there was little or insignificant degradation of zyklophin in rat blood after three hours. In contrast, arodyn exhibited a half life of <2 minutes in rat blood. Also, the half life of zyklophin was found to be 70 minutes in rat brain homogenate. In contrast, arodyn and other dynorphin A analogs exhibited a half life of ≦10-11 minutes in rat brain homogenate. Accordingly, the stability of zyklophin and lack of metabolic degradation can be important for the peptide to be able to reach its site of action in the brain intact and functional. Also, the stability and lack of metabolic degradation provides for the ability of systemic administration, especially considering zyklophin can cross the BBB.

It has now been found that zyklophin can be more potent and effective as a KOR antagonist than small molecule KOR antagonists. Surprisingly and unexpectedly, zyklophin has been found to be much more potent (e.g., 14-fold) as a KOR antagonist following systemic administration than one of the most potent small molecule KOR antagonists, JDTic. That is, zyklophin can have a higher activity level for a shorter period of time compared to JDTic. Accordingly, zyklophin can be administered in an amount lower than JDTic or other small molecule KOR antagonists. Thus, zyklophin can be useful as a KOR antagonist in the treatment, inhibition, and/or prevention of cocaine or other drug addiction, or cocaine or other drug-seeking activity, and can be prepared in composition and used in methods of administration for use in the same.

In one embodiment, zyklophin can be used for the treatment, inhibition, and/or prevention of cocaine abuse by blocking stress-induced reinstatement of cocaine-seeking behavior following systemic administration. Accordingly, zyklophin can be used as a therapeutic agent or prophylactic to inhibit or prevent the cocaine addiction activities. It is expected that zyklophin may also be active in models for treatment, inhibition, or prevention of drug abuse, drug-seeking behavior, and stress-induced drug-seeking behaviour of other drugs of abuse, specifically opiates and amphetamine, and possibly others such as alcohol, nicotine, crack, crank, ice, ecstasy, and the like. Based on the activity of small molecule KOR antagonists, zyklophin is also expected to exhibit antidepressant activity.

### III. ZYKLOPHIN COMPOSITIONS AND ADMINISTRATION

The zyklophin compound or analogs of the present invention can be formulated into a pharmaceutically acceptable formulation. Such a composition can be useful to prevent, alleviate, eliminate, inhibit or delay the onset of cocaine or other drug addiction, or cocaine or other drug-seeking activity. Accordingly zyklophin compositions can be used as a prophylactic or treatment for cocaine or other drug addiction behavior, or cocaine or other drug-seeking activity. Also, the present invention may be useful for treating and/or preventing any other type of addiction or substance or activity-seeking behavior.

In embodiments of the present invention, the pharmaceutical composition comprises at least one active component and inactive components. The active components are zyklophin compounds described herein and their derivatives/analogues. The inactive components are selected from the group consisting of excipients, carriers, solvents, diluents, stabilizers, enhancers, additives, adhesives, and combinations thereof.

Pharmaceutical preparations include sterile aqueous or non-aqueous solutions, suspensions and emulsions. Examples of non-aqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing these pharmaceutical compositions without resort to undue experimentation.

Pharmacological compositions may be prepared from water-insoluble compounds, or salts thereof, such as aqueous base emulsions. In such embodiments, the pharmacological composition will typically contain a sufficient amount of pharmaceutically acceptable emulsifying agent to emulsify the desired amount of the pharmacological agent. Useful emulsifying agents include, but are not limited to, phosphatidyl cholines, lecithin, and the like.

Additionally, the compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Furthermore, pharmacological agent compositions may, though not always, contain microbial preservatives. Microbial preservatives that may be employed include, but are not limited to, methylparaben, propylparaben, and benzyl alcohol. The microbial preservative may be employed when the pharmacological agent formulation is placed in a vial designed for multi-dose use. Pharmacological agent compositions for use in practicing the subject methods may be lyophilized using techniques well known in the art.

The compositions may also include components, such as cyclodextrins, to enhance the solubility of one or more other components included in the compositions. Cyclodextrins are widely known in the literature to increase the solubility of poorly water-soluble pharmaceuticals or drugs and/or enhance pharmaceutical/drug stability and/or reduce unwanted side effects of pharmaceuticals/drugs. For example, steroids, which are hydrophobic, often exhibit an increase in water solubility of one order of magnitude or more in the presence of cyclodextrins. Any suitable cyclodextrin component may be employed in accordance with the present invention. The useful cyclodextrin components include, but are not limited to, those materials which are effective in increasing the apparent solubility, preferably water solubility, of poorly soluble active components and/or enhance the stability of the active components and/or reduce unwanted side effects of the active components. Examples of useful cyclodextrin components include, but are not limited to: β-cyclodextrin, derivatives of β-cyclodextrin, carboxymethyl-β-cyclodextrin, carboxymethyl-ethyl-β-cyclodextrin, diethyl-β-cyclodextrin, dimethyl-β-cyclodextrin, methyl-β-cyclodextrin, random methyl-β-cyclodextrin, glucosyl-β-cyclodextrin, maltosyl-β-cyclodextrin, hydroxyethyl-β-cyclodextrin, hydroxypropyl-β-cyclodextrin, sulfobutylether-β-cyclodextrin, and the like and mixtures thereof.

The pharmaceutically acceptable excipients, such as vehicles, adjuvants, carriers or diluents, are readily available to the public. Examples of suitable excipients can include, but are not limited to, the following: acidulents, such as lactic acid, hydrochloric acid, and tartaric acid; solubilizing components, such as non-ionic, cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin, povidone, starch, vegetable oil, and the like; buffering components, such as sodium phosphate, malic acid, and potassium citrate; chelating components, such as EDTA, malic acid, and maltol; coating components, such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as colloidal silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitostearate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, polyethylene glycol, triacetin, and the like; stabilizing components, such as cyclodextrins, albumin, xanthan gum; and tonicity components, such as glycerol, dextrose, potassium chloride, and sodium chloride; and mixture thereof. Excipients include those that alter the rate of absorption, bioavailability, or other pharmacokinetic properties of pharmaceuticals, dietary supplements, alternative medicines, or nutraceuticals.

Other examples of suitable excipients, binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

In some embodiments, the compounds in the compositions may be present as a pharmaceutically acceptable salt. The pharmaceutically acceptable salts includes salts of the active agent or components of the composition, prepared, for example, with acids or bases, depending on the particular substituents found within the composition and the treatment modality desired. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, trifluoroacetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galactunoric, salicylic, formic, naphthalene-2-sulfonic, and the like. Still other suitable acids include amino acids such as arginate, aspartate, glutamate, and the like.

In general, pharmaceutically acceptable carriers are well-known to those of ordinary skill in the art. This carrier can be a solid or liquid and the type is generally chosen based on the type of administration being used. Suitable pharmaceutical carriers are, in particular, fillers, such as sugars, for example lactose, sucrose, mannitol or sorbitol, cellulose preparations and/or calcium phosphates, for example tricalcium phosphate or calcium hydrogen phosphate, furthermore, binders such as starch paste, using, for example, corn, wheat, rice or potato starch, gelatin, tragacanth, methylcellulose and/or polyvinylpyrrolidone, if desired, disintegrants, such as the above mentioned starches; furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol.

Additional pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Additional formulations for use in the present invention can be found in Remington's Pharmaceutical Sciences (Mack Publishing Company, Philadelphia, Pa., 17th ed. (1985)), which is incorporated herein by reference. Moreover, for a brief review of methods for drug delivery, see, Langer, Science 249:1527-1533 (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. Other examples of suitable pharmaceuticals are listed in 2000 Med Ad News 19:56-60 and The Physicians Desk Reference, 53rd edition, 792-796, Medical Economics Company (1999), both of which are incorporated herein by reference.

In general, compounds of this invention can be administered as pharmaceutical compositions by any one of the following routes: oral, systemic (e.g., transdermal, intranasal or by suppository), intrathecal (e.g., into the spinal canal), or parenteral (e.g., intramuscular, intravenous or subcutaneous) administration. Compositions can take the form of tablets, pills, capsules, semisolids, powders, sustained release formulations, solutions, suspensions, elixirs, aerosols, or any other appropriate compositions. Another manner for administering compounds of this invention is inhalation.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilizers.

Suitable rectally utilizable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

According to the methods of the present invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced using any mode of administration that is medically acceptable, meaning any mode that produces effective levels of the active composition without causing clinically unacceptable adverse effects.

The administration may be localized (i.e., to a particular region, physiological system, tissue, organ, or cell type) or systemic. For example, the composition may be administered through parental injection, implantation, orally, vaginally, rectally, buccally, pulmonary, topically, nasally, transdermally, surgical administration, or any other method of administration where access to the target by the composition is achieved. In one example, the administration is directly into the brain or brain cavity. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system.

For injection, the compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For administration, the compounds can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. In the present case, inhalation can be especially advantageous.

The compounds can be formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulator agents such as suspending, stabilizing and/or dispersing agents.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds can be encapsulated in a vehicle such as liposomes that facilitates transfer of the bioactive molecules into the targeted tissue, as described, for example, in U.S. Pat. No. 5,879,713 to Roth et al. and Woodle, et al., U.S. Pat. No. 5,013,556, the contents of which are hereby incorporated by reference. The compounds can be targeted by selecting an encapsulating medium of an appropriate size such that the medium delivers the molecules to a particular target. For example, encapsulating the compounds within microparticles, preferably biocompatible and/or biodegradable microparticles, which are appropriate sized to infiltrate, but remain trapped within, the capillary beds and alveoli of the lungs can be used for targeted delivery to these regions of the body following administration to a patient by infusion or injection.

Microparticles and nanoparticles can be fabricated from different polymers using a variety of different methods known to those skilled in the art. The solvent evaporation technique is described, for example, in E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984). The hot-melt microencapsulation technique is described by E. Mathiowitz, et al., Reactive Polymers, 6, 275 (1987). The spray drying technique is also well known to those of skill in the art. Spray drying involves dissolving a suitable polymer in an appropriate solvent. A known amount of the compound is suspended (insoluble drugs) or co-dissolved (soluble drugs) in the polymer solution. The solution or the dispersion is then spray-dried. Microparticles ranging between 1-10 microns are obtained with a morphology which depends on the type of polymer used.

Microparticles made of gel-type polymers, such as alginate, can be produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath to allow sufficient time for gelation to occur. Microparticle particle size is controlled by using various size extruders or varying either the nitrogen gas or polymer solution flow rates.

Embodiments may also include administration of at least one pharmacological agent using a pharmacological delivery device or system such as, but not limited to, pumps (implantable or external devices), epidural injectors, syringes or other injection apparatus, catheter and/or reservoir operatively associated with a catheter, injection, and the like. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert at least one pharmacological agent into the delivery device for administration of the pharmacological agent to a subject and include, but are not limited to, vials, ampules, tubes, capsules, bottles, syringes and bags.

Sterile injectable forms of the compositions of this invention may be aqueous or a substantially aliphatic suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

Pharmacological agents may be delivered transdermally, by a topical route, formulated as applicator sticks, solutions, suspensions, emulsions, gels, creams, ointments, pastes, jellies, paints, powders, and aerosols. For example, embodiments may include a pharmacological agent formulation in the form of a discrete patch or film or plaster or the like adapted to remain in intimate contact with the epidermis of the recipient for a period of time. For example, such transdermal patches may include a base or matrix layer, e.g., polymeric layer, in which one or more pharmacological agent(s) are retained. The base or matrix layer may be operatively associated with a support or backing. Pharmacological agent formulations suitable for transdermal administration may also be delivered by iontophoresis and may take the form of an optionally buffered aqueous solution of the pharmacological agent compound. Suitable formulations may include citrate or bis/tris buffer (pH 6) or ethanol/water and contain a suitable amount of active ingredient. Topical application for the lower intestinal tract can be effected in a rectal suppository formulation (see above) or in a suitable enema formulation. Topically-transdermal patches may also be used.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Depending on the mode of administration, the pharmaceutical composition will preferably comprise from 0.05 to 99% by weight, more preferably from 0.1 to 70% by weight of the active ingredient, and, from 1 to 99.95% by weight, more preferably from 30 to 99.9 weight % of a pharmaceutically acceptable carrier, all percentages being based on the total composition.

The compositions of the present invention may be given in dosages, generally at the maximum amount while avoiding or minimizing any potentially detrimental side effects. The compositions can be administered in effective amounts, alone or in a cocktail with other compounds, for example, other compounds that can be used to treat, inhibit, or prevent drug addiction or drug-seeking behavior.

In one embodiment of the present invention, therapeutically effective amounts of compounds of the present invention may range from approximately 0.01 to 50 mg per kilogram body weight of the recipient per day; preferably about 0.01 to 25 mg/kg/day, more preferably from about 0.05 to 10 mg/kg/day. Thus, for administration to a 70 kg person, the dosage range would most preferably be about 3 to 70 mg per day.

In another embodiment of the present invention, dosages may be estimated based on the results of experimental models, optionally in combination with the results of assays of the present invention. In the event that the response of a particular subject is insufficient at such doses, even higher doses (or effective higher doses by a different, more localized delivery route) may be employed to the extent that patient tolerance permits. Multiple doses per day are also contemplated in some cases to achieve appropriate systemic levels of the composition.

Use of a long-term release implant may be particularly suitable in some cases. “Long-term release,” as used herein, means that the implant is constructed and arranged to deliver therapeutic levels of the composition for at least 7 days, and preferably at least 14 or 30 days, or even longer in some cases. Long-term release implants are well known to those of ordinary skill in the art, and include some of the release systems described above.

Any suitable dosage may be administered. The compound, the carrier, and the amount will vary widely depending on body weight, the severity of the condition being treated and other factors that can be readily evaluated by those of skill in the art. Generally a dosage of between about 0.01 mg per kg of body weight and about 10 mg per kg of body weight is suitable.

In pharmaceutical dosage forms, agents may be administered alone or with an appropriate association, as well as in combination, with other pharmaceutically active compounds. As used herein, “administered with” means that at least one pharmacological agent and at least one other adjuvant (including one or more other pharmacological agents) are administered at times sufficiently close that the results observed are indistinguishable from those achieved when one pharmacological agent and at least one other adjuvant (including one or more other pharmacological agents) are administered at the same point in time. The pharmacological agent and at least one other adjuvant may be administered simultaneously (i.e., concurrently) or sequentially. Simultaneous administration may be carried out by mixing at least one pharmacological agent and at least one other adjuvant prior to administration, or by administering the pharmacological agent and at least one other adjuvant at the same point in time. Such administration may be at different anatomic sites or using different routes of administration. The phrases “concurrent administration,” “administration in combination,” “simultaneous administration” or “administered simultaneously” may also be used interchangeably and mean that at least one pharmacological agent and at least one other adjuvant are administered at the same point in time or immediately following one another. In the latter case, at least one pharmacological agent and at least one other adjuvant are administered at times sufficiently close that the results produced are synergistic and/or are indistinguishable from those achieved when at least one pharmacological agent and at least one other adjuvant are administered at the same point in time. Alternatively, a pharmacological agent may be administered separately from the administration of an adjuvant, which may result in a synergistic effect or a separate effect. The methods and excipients described herein are merely exemplary and are in no way limiting.

Therapeutically effective dosages for the compounds described herein can be estimated initially from cell culture assays. For example, a dose can be formulated in animal models to achieve a circulating concentration range that includes the IC₅₀ as determined in cell culture (i.e., the concentration of test compound that is effective to 50% of a cell culture), or the IC₁₀₀ as determined in cell culture (i.e., the concentration of compound that is effective in 100% of a cell culture). Such information can be used to more accurately determine useful doses in humans. Initial dosages can also be estimated from in vivo data.

Moreover, toxicity and therapeutic efficacy of the compounds described herein can be determined by standard pharmaceutical procedures in cell cultures or experimental animals, e.g., by determining the LD₅₀, (the dose lethal to 50% of the population), the ED₅₀ (the dose therapeutically effective in 50% of the population), and EC₅₀ (the effective concentration effective in 50% of the population). The dose ratio between toxic and therapeutic effect is the therapeutic index and can be expressed as the ratio between LD₅₀ and ED₅₀. Compounds which exhibit high therapeutic indices are candidates for further development. The data obtained from these cell culture assays and animal studies can be used in formulating a dosage range that is not toxic for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: “The Pharmacological Basis of Therapeutics”, Ch. 1, p. 1). Additionally, the EC₅₀ can be important to measure.

Dosage amount and interval may be adjusted individually to provide plasma levels of the active compound which are sufficient to maintain therapeutic effect. In cases of local administration or selective uptake, the effective local concentration of the drug may not be related to plasma concentration. One having skill in the art will be able to optimize therapeutically effective local dosages without undue experimentation.

In one embodiment, a catheter can be used to direct the composition directly to the brain or other location in the body for systemic delivery. As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact formulation, route of administration and dosage for the pharmaceutical compositions of the present invention can be chosen by the individual physician in view of the patient's condition. Typically, the dose range of the composition administered to the patient can be from about 0.05 to 20 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from EC₅₀, ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will vary dependent upon the percent composition of the dosage of compounds of the present invention, in most cases some generalizations regarding the dosage can be made. The daily dosage regimen for an adult human patient may be, for example, a dose of between 0.1 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. The dosage per weight can be up to about 3 mg/kg, or between 1 ug/kg to about 3 mg/kg, more preferably from about 10 ug/kg to about 2 mg/kg, or between 100 ug/kg to about 750 ug/kg. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the compounds disclosed herein in amounts that exceed, or even far exceed, the above-stated dosage range in order to effectively and aggressively treat particularly aggressive diseases. In some embodiments, the compounds will be administered for a period of continuous therapy, for example for a week or more, or for months or years.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

### IV. EXPERIMENTAL SECTION

### A. Antinociceptive Testing and Intracerebroventricular Injection Technique

The 55° C. warm-water tail-withdrawal assay was used as described earlier (McLaughlin et al., 1999), with the latency of the mouse to withdraw its tail taken as an endpoint. After determining baseline tail-withdrawal latencies, mice received a single dose of vehicle or zyklophin and were allowed to recover. After recovery, a single dose of the kappa-opioid receptor agonist, U50,488 (10 mg/kg, i.p.) was administered. The dose of U50,488 was selected based on previous demonstration of significant kappa-opioid mediated antinociception in C57B1/6J mice (McLaughlin et al., 2006). Mice administered U50,488 were subsequently tested 40 min later for their tail-withdrawal latencies to determine the duration of kappa-opioid receptor antagonism produced by zyklophin. The initial dose of zyklophin examined was selected based on the previous characterization of arodyn (see Carey et al., 2007).

### B. Cocaine-Conditioned Place Preference, Extinction, and Reinstatement

The conditioned place preference assay was performed as described herein. C57B1/6J mice were conditioned using a protocol similar to the previously established biased cocaine-conditioned place preference paradigm (Szumlinski et al., 2002; McLaughlin et al., 2003 and 2006; Carey et al., 2007). The apparatus was a compartmentalized box divided into two equal-sized outer compartments (25 cm×25 cm×25 cm) with distinct cues, each joined to a small central section (8.5 cm×25 cm×25 cm) accessed through a single doorway (3 cm high). The entire unit was fitted with infrared beams, the breaking of which allows an automated measure of the time animals spend in each chamber (San Diego Instruments, San Diego, Calif., USA). The compartments differ in wall striping (vertical vs. horizontal alternating black and white lines, 1.5 cm in width) and floor texture (lightly mottled vs. smooth). Note that the biased place-conditioning protocol involves administration of cocaine to mice in the outer compartment opposite of their preference response in an initial preconditioning preference test. The biased conditioned place preference protocol produces a sensitive indicator of conditioned drug reward that is consistent across studies (Shimosato and Ohkuma, 2000; Szumlinski et al., 2002, McLaughlin et al., 2003 and 2006), equivalent to alternative methods (e.g., the counterbalanced design; see Bardo et al., 1995 for a review). Moreover, the biased conditioned place preference design has the advantage of controlling for the individual animal's bias in the apparatus, allowing for the more efficient use of the animals available. It has also been demonstrated as an effective protocol for the study of extinction and reinstatement (Szumlinski et al., 2002).

Time spent in each compartment was measured by allowing individual mice to move freely between all three compartments over a 30-min testing period. Place-conditioning began immediately following cocaine administration (10 mg/kg, s.c.), whereupon mice were consistently confined for 30 min in the appropriate outer compartment. A dose of 10 mg/kg s.c. cocaine was selected for this study as it has been shown previously to produce a reliable conditioned-place preference response in C57B1/6J mice (Zhang et al., 2002; Kreibich and Blendy, 2004; Brabant et al., 2005). Conditioning with assay vehicle (0.9% saline, 0.3 ml/30 g body weight, s.c.) followed 4 h later in a similar manner, but paired to the opposite chamber. This conditioning cycle was repeated once on each of four days, which has been demonstrated to be effective in maintaining the place preference response for approximately 3 weeks (Brabant et al., 2005). Data are plotted as the difference in time spent in the eventual cocaine- and vehicle-paired compartments, such that by convention the initial bias generates a negative value, and a positive value reflects a conditioned preference for the cocaine-paired side.

The conditioned place preference extinction assay was performed as described herein. Place preference for the cocaine-paired compartment was re-examined weekly to confirm extinction. Placing animals repeatedly into the apparatus with free access to all compartments for 30 min produced extinction, defined as a statistically significant decrease in the time spent in the cocaine-paired compartment during the extinction trial as compared to the immediate postconditioning response. As expected for the C57B1/6J strain of mice, conditioned place preference responses subsided with repeated testing over the three week period (Szumlinski et al., 2002; Kreibich and Blendy, 2004; Brabant et al., 2005; Carey et al., 2007).

The conditioned place preference reinstatement assay was performed as described herein. Reinstatement of drug preference was examined after either exposure to forced swim stress (see section C, below) or an additional cycle of cocaine place-conditioning. Note that a single cycle of cocaine place-conditioning was found to be insufficient to produce conditioned place preference alone in C57B1/6J mice (Brabant et al., 2005; Carey et al., 2007). Mice were pretreated s.c. with vehicle or zyklophin one hour prior to either cocaine conditioning or forced swimming on the first day. The day after completion of stress exposure or cocaine conditioning, animals were tested for place preference.

### C. Forced Swim Stress

A two-day forced swim stress protocol was used as previously detailed (McLaughlin et al., 2003) to produce stress-induced reinstatement of cocaine-conditioned place preference. Mice were pretreated daily with vehicle or zyklophin one hour prior to forced swimming. One hour after the final exposure to forced swim stress, the place preference responses of mice were tested as described above to determine possible reinstatement of extinct conditioned place preference.

### D. Synthesis

Zyklophin was synthesized as described elsewhere (Patkar et al., 2005).

### E. Cell Culture

An in-vitro model of the blood-brain barrier (BBMEC) was employed to determine the permeability of Dyn A analogs. BBMEC cells were isolated as described by Audus et al (Audus et al, 1996). Cells were seeded at a density of 50,000 cells/cm² using plating media (50% Ham's F-12 nutrient mixture, 50% MEM, 10 mM HEPES, 13 mM sodium bicarbonate, penicillin G (100 μg/ml), streptomycin (100 μg/ml) and 10% platelet poor horse serum) on a sterile petri dish containing polycarbonate membranes (Nuclepore Track-etch, PC MB, 13 mm) coated with collagen and fibronectin. Media was changed every 48 hrs with changing media (‘plating media’+endothelial cell growth factor and heparin) until the cells attained confluency (10-14 days).

### F. Transport Studies

Transport studies were conducted using polycarbonate membranes mounted on silanized Side-bi-Side™ diffusion cells (Crown Glass Co., Somerfield, N.J.) (Chappa et al, 2006). Transport of Dyn A or its analogs was conducted in a buffer made of 50% Ham's F-12 and 50% MEM adjusted to pH 7.4. The donor and receiver chambers were sampled at various time intervals for a period of 2 hrs after adding Dyn A or its analogs at time ‘0’. Samples were then spiked with a known concentration of the internal standard and analyzed by LC-MS/MS. The samples were then monitored for the appearance of parent peptides as well as metabolites. At the end of the study, the integrity of the monolayer was determined by evaluating the permeability of the paracellular marker, ¹⁴C-sucrose. The permeability of DynA analogs was calculated using the following equation: Pₐₚₚ=(ΔQ/Δt)/A×Co, where ΔQ/Δt=linear appearance rate of Dyn A or its analogs in the receiver chamber, A=cross-sectional area (0.636 cm²) and Cₒ=initial concentration of the test compound in donor chamber at t=0.

### G. LC-MS/MS

Optimal separation of Dyn A or its analogs was achieved on a Vydac Microbore C-18 MS column (5 μm, 1 mm ID×15 mm OD) at a flow rate of 0.2 ml/min using a mobile phase gradient of 0 to 60% acetonitrile in 0.1% formic acid. MS/MS detection was performed in ESI+ mode on a Micromass QuattroMicro™ instrument coupled with Waters 2690 solvent delivery system. Samples were analyzed by spiking them with appropriate internal standard.

### Example 1

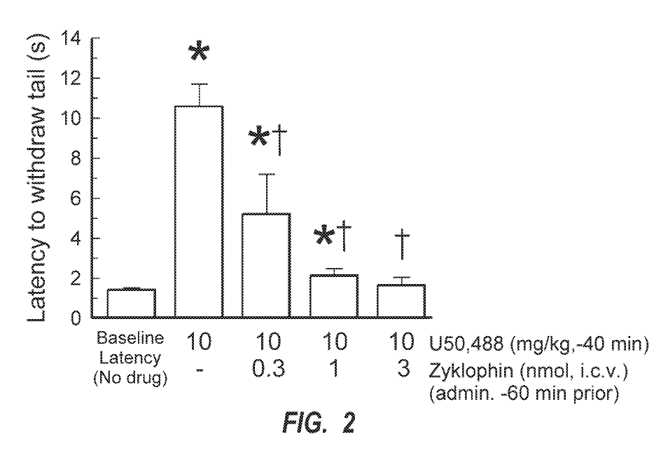

Zyklophin was examined for its ability to antagonize the analgesic activity of the KOR agonist U50,488 in the 55° C. warm water tail withdrawal assay in order to determine effective doses to use in subsequent assays. Initially, the zyklophin peptide was administered directly into the brain (e.g., intracerebroventricularly, i.c.v.,) to establish KOR antagonist activity. As shown in FIG. 2, zyklophin can reverse the effects of U50,488 in a dose-dependent manner, thereby verifying that it exhibits KOR antagonist activity in vivo. Zyklophin was administered intracerebroventricularly at 0.3, 1, or 3 nmol/mouse, and U50,488 was administered intraperitoneally (i.e., i.p.).

Baseline tail-withdrawal responses were first characterized for all mice (left-most bars of each figure). Antinociceptive effects of U50,488 (10 mg/kg, i.p.) in mice pretreated 1 h with zyklophin through the (FIG. 2) i.c.v. route of administration. Tail-withdrawal latency was measured in the mouse 55° C. warm-water tail-withdrawal test at 40 min after the injection of the U50,488. Bars each represent n=6-8 mice.

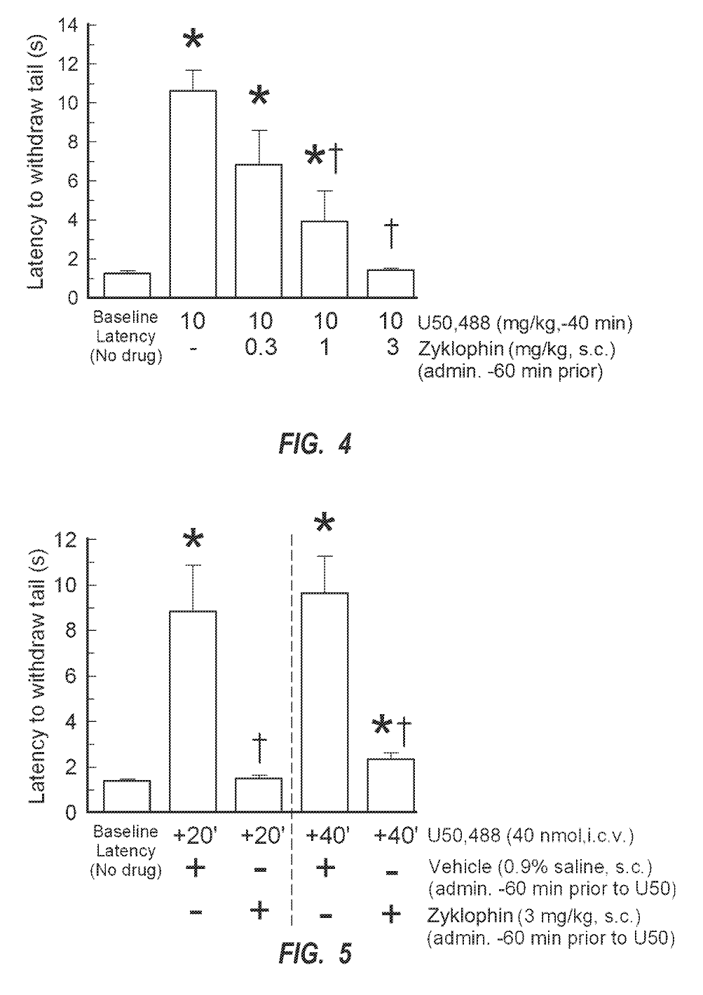

Initial tests demonstrated that zyklophin lacked antinociceptive effect, as neither i.c.v. (3 nmol) nor s.c. administration (1 or 3 mg/kg) significantly changed the baseline tail-withdrawal latency up to 60 min later (1.42±0.14 s baseline latency versus 1.84±0.41 s latency after i.c.v. administration, and 1.48±0.16 s baseline latency versus 1.73±0.15 s and 1.4±0.08 s latency after 1 and 3 mg/kg zyklophin s.c., respectively). Mice administered U50,488 showed significant increases in tail-withdrawal latency (FIG. 2). Intracerebroventricular pretreatment with zyklophin (0.3, 1 or 3 nmol) 1 h prior to testing significantly antagonized the antinociceptive effect of the KOR-selective agonist U50,488 (FIG. 2). Notably, peripheral pretreatment with zyklophin (1 or 3 mg/kg s.c.) 1 h prior to testing also significantly antagonized the antinociceptive effect of U50,488 (FIG. 4). Importantly, peripheral administration of zyklophin (3 mg/kg, s.c.) 1 h prior to testing antagonized the antinociceptive effect of U50,488 administered centrally (40 nmol i.c.v., FIG. 5) 20 min earlier, which is strong evidence that the peptide antagonist crossed the BBB in vivo to act on KOR in the brain.

### Example 2

In a subsequent assay the peptide was administered systemically (e.g., subcutaneously). Zyklophin was administered subcutaneously to reverse the analgesic activity of U50,488. More particularly, zyklophin was administered subcutaneously at 0.3, 1 or 3 mg/kg, and U50,488 was administered intraperitoneally. The data of Figure 4 indicates that the zyklophin peptide is systemically active. Such activity is obtained by zyklophin traversing the BBB with sufficient activity to function as a KOR antagonist. Additional discussion is found in Example 1 above.

### Example 3

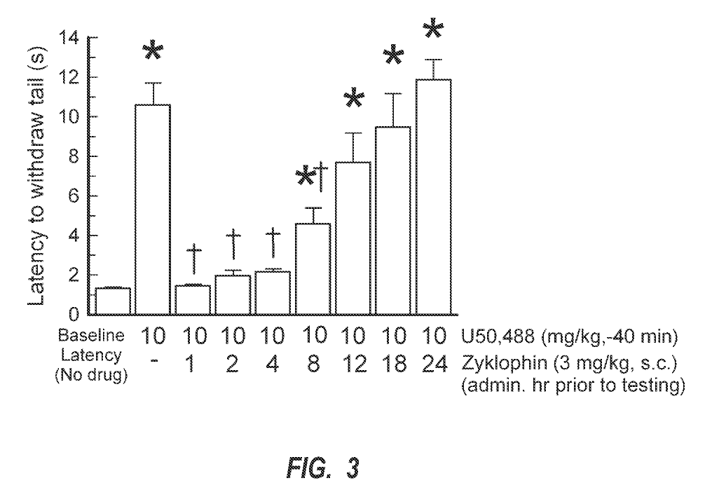

Additionally, data shown in FIG. 3 confirmed the duration of in vivo kappa-opioid receptor antagonist effects of zyklophin in C57B1/6J mice using the 55° C. warm-water tail-withdrawal test. A number of nonpeptide kappa-opioid receptor-selective antagonists, such as nor-binaltorphimine and JDTic, demonstrate a prolonged duration of action (Horan et al., 1992). As such, the duration of kappa-opioid receptor antagonism produced by a single dose of zyklophin was studied. Mice were pretreated through the subcutaneous route with zyklophin (3 mg/kg, s.c.) 80 min to 23.3 (24 h) in advance of an intraperitoneal administration of U50,488 (10 mg/kg), and antinociception measured in the 55° C. warm-water tail-withdrawal test (FIG. 3). As shown, intraperitoneal administration of the kappa-opioid receptor agonist U50,488 (10 mg/kg) produced significant antinociception 40 min after administration (FIG. 3, second bar from left), significantly greater than the baseline tail-withdrawal latency (FIG. 3, left bar). However, consistent with the previous data of zyklophin (FIG. 4), subcutaneous pretreatment with zyklophin at 3 mg/kg at 1 h prior to testing significantly antagonized the antinociceptive effect of U50,488 (FIG. 3, third bar from left; as compared to U50,488 alone). Thus, zyklophin pretreatment antagonized U50,488-induced antinociception for at least 4 hours, with a partial recovery at 8 h and a full recovery by 18 h. These findings demonstrate a brief duration of kappa-opioid receptor antagonism produced by zyklophin, in contrast to established nonpeptide kappa-opioid receptor antagonists (Horan et al., 1992; Carroll et al., 2004).

FIG. 3 shows that zyklophin significantly antagonizes U50,488 for at least 8 hours, but less than 12 hours. Since small molecule KOR antagonists are usually active for significantly more than 12 hours, such as a day or more, zyklophin has a shorter half life and is likely to have less negative side effects and more controllability over administration and obtaining a desired therapeutic effect. Thus, zyklophin pretreatment significantly antagonized U50,488-induced antinociception for at least 8 h, but for less than 12 h. These findings demonstrate a reversible, relatively short duration of KOR antagonism produced by zyklophin, unlike established KOR-selective nonpeptide antagonists which exhibit exceptionally long activity after a single dose.

### Example 4

It is possible that U50,488 can produce some of its analgesic activity by interacting with KOR in the periphery rather than in the brain. Therefore to verify that zyklophin was crossing the blood-brain barrier and antagonizing KOR in the brain, U50,488 was administered directly into the brain (e.g., i.c.v.). More particularly, zyklophin was administered subcutaneously at 3 mg/kg, and U50,488 was administered intracerebroventricularly at 40 nmol/mouse. FIG. 5 shows that zyklophin administered subcutaneously blocked the agonist activity of intracerebroventricularly administered U50,488, and thus zyklophin does cross into the brain and does have the ability to reverse the activity of U50,448. The ability of subcutaneously administered zyklophin to antagonized centrally administered U50,488 demonstrates that zyklophin is active centrally following systemic administration

### Example 5

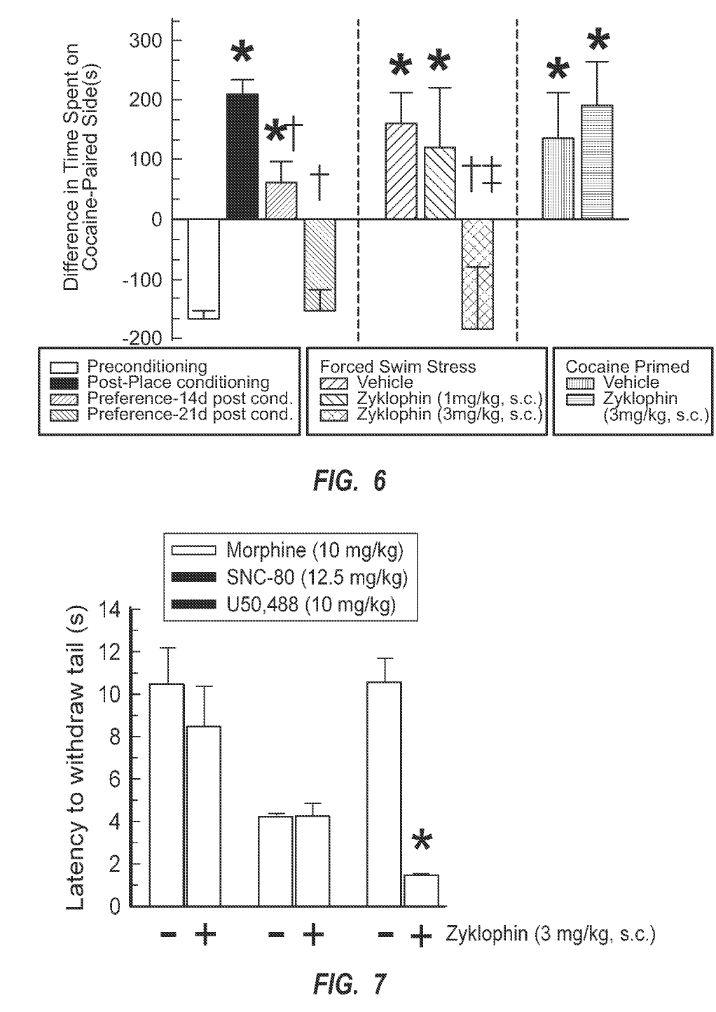

The kappa-opioid receptor antagonist JDTic was previously reported to suppress stress-induced reinstatement of cocaine self-administration (Beardsley et al., 2005). The peptide zyklophin acts as a kappa-opioid receptor antagonist and was studied to see if it can also prevent stress-induced reinstatement of cocaine seeking behavior. To study this, C57B1/6J mice were first place conditioned over four days with cocaine (10 mg/kg s.c. daily). Mice demonstrated a cocaine-conditioned place preference that was significantly greater than that of the initial preference response (FIG. 6 left bars; F_{(4,375)}=34.3). This place preference lasted over 2 weeks (FIG. 6). After 3 weeks, mice demonstrated extinction with a place preference response that was statistically less than the initial postconditioning preference (FIG. 6).

Following extinction of cocaine-CPP, mice were administered vehicle (0.9% saline) or the peptide KOR antagonist zyklophin (1 or 3 mg/kg, s.c.) daily and exposed to repeated forced swim stress. After exposure to stress on the second day, mice were tested for place preference to examine possible reinstatement of drug seeking behavior. Stress-exposed vehicle-pretreated mice subsequently demonstrated reinstatement of CPP (FIG. 6, F_{(6,292)}=22.8). In contrast, zyklophin pretreatment dose-dependently prevented stress-induced reinstatement. Mice pretreated daily with 3, but not 1, mg/kg s.c. zyklophin prior to exposure to forced swimming demonstrated place preference responses that did not differ significantly from preconditioning or extinction responses (FIG. 6). Notably, daily treatment with zyklophin (3 mg/kg, s.c.) immediately after exposure to forced swimming did not prevent stress-induced reinstatement (FIG. 6). In addition, mice demonstrating extinction of CPP were subsequently exposed to a single round of cocaine conditioning prior to place preference testing. Cocaine-exposed mice pretreated with vehicle exhibited reinstatement of place preference. Mice treated with zyklophin (3 mg/kg, s.c.) before exposure to an additional cocaine conditioning cycle showed a significantly greater preference for the cocaine-paired compartment as compared to pre-conditioning and extinct preferences. Furthermore, the reinstated preference of zyklophin pretreated mice was not significantly different than the response of vehicle pretreated mice. Thus zyklophin pretreatment had no effect on cocaine-induced reinstatement of place preference. Overall, these results confirm a mediating role for the endogenous KOR system in stress-induced relapse of drug seeking behavior, as pretreatment with the novel peptide KOR antagonist zyklophin prevented the stress-induced reinstatement.

### Example 6

The KOR-selective antagonism by zyklophin was studied. As such, morphine and SNC-80, which are active with opioid receptors (e.g., mu-opioid receptor and delta-opioid receptor, respectively) other than kappa were provided to mice that had previously been treated with zyklophin. Also, U50,488, which is selective for the KOR was used as a control. As shown in FIG. 7, the antinociceptive effects of morphine (10 mg/kg, i.p.) or SNC-80 (12.5 mg/kg, i.p.) were not reduced by a 1 h pretreatment with zyklophin (3 mg/kg, s.c.), while the effect of U50,488 (10 mg/kg, i.p.) was significantly antagonized. This shows that zyklophin is selective for KOR over other opioid receptors, such as the mu-opioid receptor and the delta-opioid receptor.

### Example 7

Several Dyn A-(1-11) amide analogs were examined for their ability to cross the bovine brain microvessel endothelial cell (BBMEC) model of the BBB and for their stability to metabolic degradation; tandem mass spectrometry following HPLC separation was used to quantify the peptides. All of the peptides crossed the BBMEC model at least as well as the parent peptide Dyn A-(1-11) amide (Table 1); the cyclic analog cyclo[D-Asp⁵,Dap⁸]Dyn A-(1-11) amide exhibited 3-fold higher transport across the BBMEC than the parent linear peptide. Thus these peptides can cross the BBB to reach KOR in the central nervous system (CNS).

### Example 8

The metabolic stability of the peptides depends on the modifications to the peptides. Peptides with both N- and C-terminal modifications exhibit a half life in rat plasma of 1.5-2 hours (Table 2). As expected, peptides without N-terminal modification were rapidly metabolized (Table 2), presumably by aminopeptidases. N- and C-terminal modifications, however, were not sufficient to prevent metabolism of the peptides in rat brain homogenate, and all of the peptides were rapidly degraded in this assay (t_{1/2}≦10-11 minutes). Homogenization is likely to rupture cell membranes, releasing intracellular proteases that could metabolize these peptides, so that this system may not accurately reflect the metabolism that occurs in vivo. Endopeptidases capable of metabolizing Dyn A are also present on red blood cells, and can rapidly metabolize some Dyn A-(1-11) amide analogs, e.g. arodyn.

TABLE 1
BBMEC permeability of selected dynorphin
A-(1-11) amide analogs

| Dyn A-(1-11) amide | Permeability | Relative | Efflux |
|---|---|---|---|
| analog | (cm/sec) × 10⁶ | to Dyn A | ratio ^{a} |
| Arodyn | 7.6 ± 3.0 | 2.0 | 1.6 |
| cyclo[D-Asp⁵,Dap⁸] | 12.2 ± 2.7 | 3.2 | 1.1 |
| [N-BenzylTyr¹] | 4.1 ± 0.6 | 1.1 | 2.6 |
| Ac-Dyn A-(1-11)NH₂ | 7.1 ± 2.3 | 1.9 | 2.4 |
| Dyn A-(1-11)NH₂ | 3.8 ± 1.3 | 1.0 | 0.9 |

^{a} Permeability (basal to apical)/permeability (apical to basal)

TABLE 2
Metabolic stability of selected dynorphin A-(1-11) amide analogs

| Dyn A-(1-11) amide | Rat plasma |
|---|---|
| analog | t_{1/2} (min) |
| Arodyn | 104 ± 4 |
| cyclo[D-Asp⁵,Dap⁸] | <2 ^{a} |
| [N-BenzylTyr¹] | 90 ± 19 |
| Ac-Dyn A-(1-11)NH₂ | 111 ± 17 |
| Dyn A-(1-11)NH₂ | <2 ^{a} |

^{a} Substrates for aminopeptidases

### Example 9

Because arodyn is subject to rapid metabolism by endopeptidases in rat brain homogenate and whole blood (data not shown), we examined [N-benzylTyr¹,cyclo(D-Asp⁵,Dap⁸)]Dyn A-(1-11) amide (zyklophin). Preliminary metabolism studies show that this cyclic peptide exhibits substantially greater metabolic stability than the linear peptide arodyn in rat brain homogenate, with a half life of 70 minutes, and is stable in whole blood for at least three hours.

Accordingly, dynorphin A analogs containing 11 amino acids with molecular weights of approximately 1500 can cross the BBB to reach KOR in the CNS. While N- and C-terminal modified analogs exhibit enhanced metabolic stability, they are still prone to metabolism by endopeptidases. The cyclic Dyn A analog [N-benzylTyr¹,cyclo(D-Asp⁵,Dap⁸)]Dyn A-(1-11) amide exhibits enhanced metabolic stability compared to the linear peptides. This peptide antagonizes central KOR in vivo following peripheral administration (s.c.). Thus peptide KOR ligands have potential as therapeutic agents, with [N-benzylTyr¹,cyclo(D-Asp⁵,Dap⁸)]Dyn A-(1-11) amide representing an important lead peptide.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references, publications, journal articles, patents, published patent applications, and the like that are disclosed herein are incorporated herein by specific reference in their entirety.

What is claimed is:

1. A method for antagonizing kappa-opioid receptors present in human or animal tissue in vitro or in vivo, the method comprising:
administering an effective amount of a polypeptide kappa-opioid receptor (KOR) antagonist to a subject such that a sufficient amount of the polypeptide KOR antagonist is active in the brain for antagonizing a kappa-opioid receptor.

2. A method as in claim 1, wherein the polypeptide KOR antagonist is administered systemically.

3. A method as in claim 1, wherein the polypeptide KOR antagonist is a dynorphin-A analogue.

4. A method as in claim 1, wherein the KOR antagonist includes a cyclic peptide portion.

5. A method as in claim 1, wherein the KOR antagonist is [N-benzylTyr¹,cyclo(D-Asp⁵,Dap⁸)]Dyn A-(1-11) amide, salt, prodrug, or derivative thereof.

6. A method as in claim 1, wherein the polypeptide KOR antagonist is selective for KOR over other opioid receptors.

7. A method as in claim 1, wherein the KOR antagonist is more effective as a KOR antagonist compared to JDTic.

8. A method as in claim 1, wherein the polypeptide KOR antagonist is capable of crossing the blood brain barrier.

9. A method as in claim 1, wherein the effective amount of KOR antagonist is sufficient for treating, inhibiting, and/or preventing at least one of the following:
depression;
drug seeking behavior;
opiate seeking behavior or addiction;
methamphetamine seeking behavior or addiction;
alcohol seeking behavior or addiction;
nicotine seeking behavior or addiction;
ecstasy seeking behavior or addiction; or
cocaine and/or cocaine derivative seeking behavior or addiction.

10. A method as in claim 1, wherein the effective amount of KOR antagonist is sufficient for treating, inhibiting, and/or preventing stress-induced seeking behavior of an addictive substance selected from the group consisting of opiates, methamphetamines, alcohol, nicotine, ecstasy, cocaine, cocaine derivative, or combinations thereof.

11. A method for treating, inhibiting, and/or preventing cocaine and/or cocaine derivative seeking behavior and/or addiction, the method comprising:
identifying a person that has cocaine and/or cocaine derivative seeking behavior; and
administering an effective amount of a polypeptide kappa-opioid receptor (KOR) antagonist to the person such that a sufficient amount of the polypeptide KOR antagonist is active in the brain for antagonizing KOR.

12. A method as in claim 11, wherein the polypeptide KOR antagonist is administered systemically and crosses the blood brain barrier.

13. A method as in claim 11, wherein the polypeptide KOR antagonist is a cyclic peptide and dynorphin-A analogue.

14. A method as in claim 11, wherein the KOR antagonist is [N-benzylTyr¹,cyclo(D-Asp⁵,Dap⁸)]Dyn A-(1-11) amide, salt, prodrug, or derivative thereof.

15. A method as in claim 11, wherein the cocaine and/or cocaine derivative seeking behavior is stress-induced.

16. A method as in claim 15, further comprising:
providing a pharmaceutically-acceptable composition suitable for human administration that contains [N-benzylTyr¹,cyclo(D-Asp⁵,Dap⁸)]Dyn A-(1-11) amide and a pharmaceutically-acceptable carrier; and
systemically administering an effective amount of the [N-benzylTyr¹,cyclo(D-Asp⁵,Dap⁸)]Dyn A-(1-11) amide to the person such that a sufficient amount of the [N-benzylTyr¹,cyclo(D-Asp⁵,Dap⁸)]Dyn A-(1-11) amide crosses the blood brain barrier and is active in the brain for antagonizing kappa-opioid receptors so as to inhibit stress-induced cocaine and/or cocaine derivative seeking behavior.

17. A method as in claim 11, wherein the amount of KOR antagonist is insufficient for substantial interaction with other opioid receptors.

18. A pharmaceutical composition comprising:
a pharmaceutically-acceptable carrier; and
a therapeutically effective amount of a kappa-opioid receptor (KOR) antagonist that antagonizes a sufficient amount of KOR receptors for treating, inhibiting, and/or preventing cocaine and/or cocaine derivative seeking behavior and/or addiction.

19. A pharmaceutical composition as in claim 18, wherein the pharmaceutically acceptable carrier is suitable for systemic administration.

20. A pharmaceutical composition as in claim 18, wherein the KOR antagonist includes a dynorphin-A analogue.

21. A pharmaceutical composition as in claim 20, wherein the KOR antagonist is [N-benzylTyr¹,cyclo(D-Asp⁵,Dap⁸)]Dyn A-(1-11) amide, salt thereof, prodrug thereof, and/or derivative thereof.
